(19) United States
(12) Patent Application Publication
(10) Pub. No.: US 2013/0035558 A1
(43) Pub. Date: Feb. 7, 2013

(54) SUBJECT VITALITY INFORMATION SYSTEM

(71) Applicant: Iven Jon Mestry, Shakopee, MN (US)

(72) Inventor: Iven Jon Mestry, Shakopee, MN (US)

(21) Appl. No.: 13/204,284

(22) Filed: Aug. 5, 2011

(51) Int. Cl. A61B 5/00

(52) U.S. Cl. 600/301; 600/300

(57) ABSTRACT

Instead of requiring interested parties to actively watch monitored subjects continuously and be present with them or nearby, take notes of behavior, etc., this system generates processed and summary information using sensor(s) and/or other data input sources. The information can be pushed to the recipients, allowing them to be away and/or be working on other activities. Recipients of subject vitality information receive periodic reports and/or alerts of activity, wellness and environmental events. In addition, this system also can generate intelligent assessments and/or diagnostics of subjects based on all data input sources, history, subject profiles and user entered rules.

This is an assisting solution to enhance care and should not be used as a substitute for primary care/supervision for persons whose lives may be at risk (small children and persons with severe life threatening conditions).

### TECHNICAL FIELD

The subject vitality information system is used primarily to assist care givers, keeping them aware of the wellness of care recipients and informing them of issues needing immediate attention. A second application is for assisting scientists studying animal species in their environment.

### BACKGROUND

For those who do not have, cannot afford or do not want/need a dedicated care giver present at all times, a Subject Vitality Information System is an assistive tool providing useful actionable information but primarily peace of mind and a sense of control to those responsible for caring for someone in need. Care givers often struggle to fit the need for supervision of loved ones with their other responsibilities and ongoing lives. With this tool they gain a new level of awareness of the daily lives of their loved ones while still able to attend to other parts of their lives. Other care enhancing tools can leverage subject vitality information to better serve care givers and their recipients.

The Vitality Information System can also be used for self-monitoring. For persons interested in identifying patterns in their own activities or to get a better understanding of the risks that they face.

### PRIOR ART

U.S. Pat. Nos.:
U.S. Pat. No. 7,884,727-Wireless occupancy and day-light sensing
U.S. Pat. No. 7,890,957-Remote management of an electronic presence
U.S. Pat. No. 7,411,510-Internet-based informal care delivery system
U.S. Pat. No. 7,808,391-Remote caregiver support system

Systems referred to as telemetry, telematics, machine-to-machine (M2M) or remote monitoring have been used to collect sensor information and transmit it. These systems have been used to monitor machines, buildings, tanks, wells, energy, location, etc. They are also being used to monitor specific medical conditions such as heart arrhythmia. Such systems can be used as input to a Subject Vitality Information System.

Systems used to manage communications channels from a single access point are also common. These would allow communication through email, fax, SMS, Twitter, XML, web services, etc to be dispatched from a single source of information. Such a system can be used as an output to the Subject Vitality Information System.

DESCRIPTION

### Description of Figures

FIG. 1-The output of the vitality information system communicates relevant, useful and important events, historical summaries and diagnostics to the recipients in the areas of subject mood, body vitals, activity and environmental risks factors.

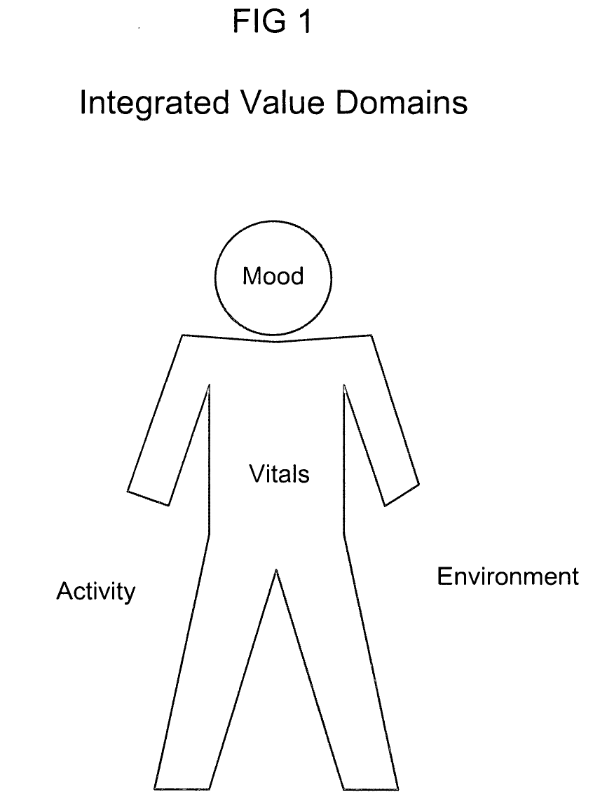

FIG. 2-The vitality information system accepts inputs from diverse sources of data and outputs timely information to communications systems. The system comprises a profile generator and a vitality engine. The profiles generator creates baselines for each subject used to extract relevant information from large amounts of data. The vitality engine coordinates the entire process of receiving input data, extracting relevant information, analyzing it using internal and external algorithms and producing formatted ‘consumable’ information about subject vitality for the recipients.

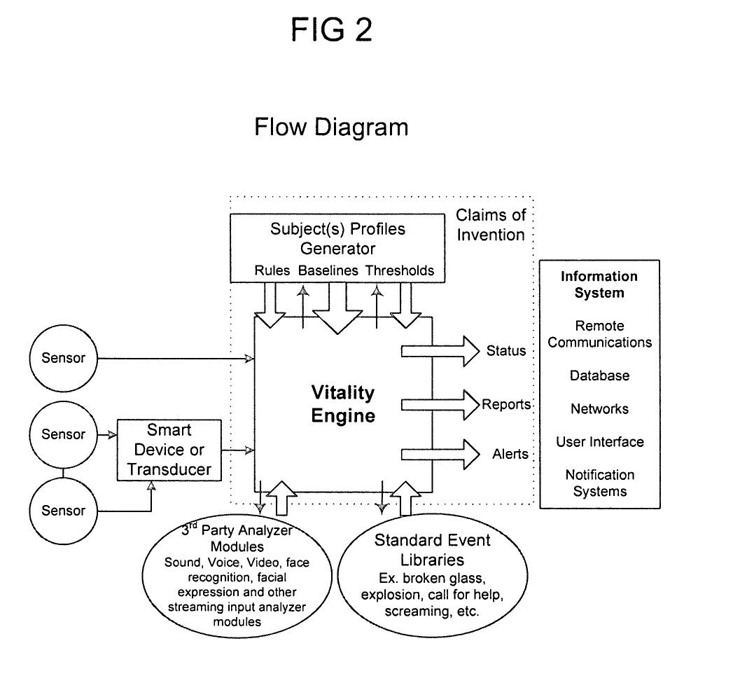

Motion Detection Analyzer

FIG. 3-A simple motion detector or a plurality of motion detectors used together can provide a wealth of information about vitality. By analyzing frequency of movement from the detectors, contact closures or other type of motion detection signals, it can be determined how active a person is or how many people there are. Signals of motion in different rooms also show motion activity from room to room.

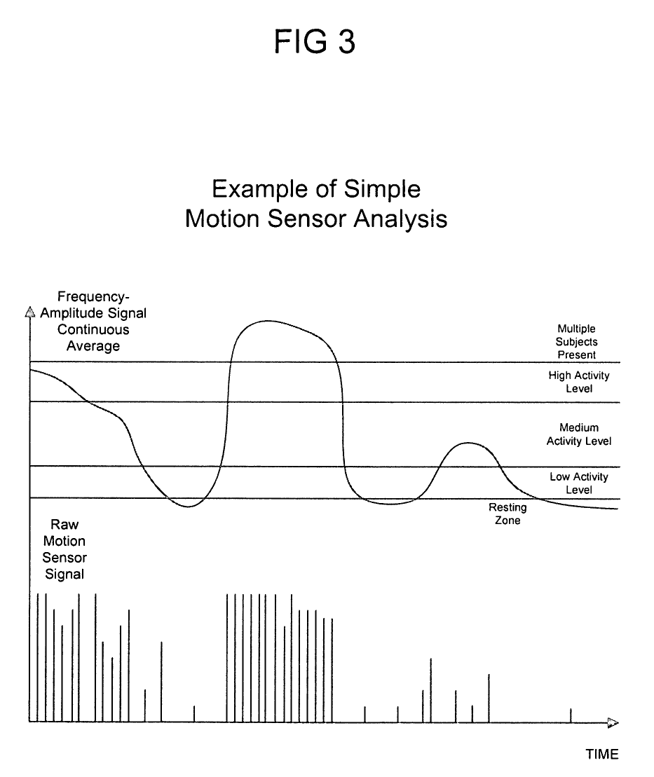

### DETAILED DESCRIPTION

Vitality Engine

Signals from sensors and input devices are classified by (1) the probability that a detected threshold would actually be an event of interest, (2) the degree of interest of the event, (3) correlation between simultaneously detected thresholds from different sources and an event.

Examples of sensors used as input for vitality engine:
1. Motion detectors, heart rate pulse counters, GPS position sensors,
2. Smart devices such as smartphones which include gyroscope, accelerometers, light sensors, etc,
3. Body temperature, blood sugar level, adrenaline, etc,
4. Door, window, entry system, perimeter fences
5. Appliance sensors and controllers, fire/smoke/CO alarms
6. Upright or lying down position
7. Brain activity sensors
8. Vehicle telematics information
9. House controller, media center, home robots,
10. Video feeds, audio feeds
11. Self-powered wireless short range sensors
12. Location or proximity sensors detecting subject leaving safe zone

Other inputs to vitality engine—voice recognition software that identifies and separates individual voices. Video analysis software.

### Examples

Multiple motion sensors in different zones defining a monitored space: ex. house with sensors in each room.
1. The signal frequency range: 0-60 cycles per minute
2. 0 cpm=resting or away from zone
3. 0<resting=<10
4. 10<low activity level=<20 (ex. watching TV, reading book)
5. 20<normal activity level=<40 (ex. cleaning, cooking, talking on the phone)
6. 40<high activity level=<60 (ex. aerobics)
7. 50<more than one person may be present

At the end of a day, a summary of activity can be sent out:
1. Compare with average day
2. Compare with normal for age
3. Compare with profile of normal as described by monitored individual (“I had a good day today”)
4. Compare with what is observed through other means

Acoustics

Sounds and voice can be used to determine a need for help. Voice recognition software can be used to detect a call for help or frustration or good mood (singing). Tapping or clapping sounds can also be used to communicate a message. Recipients of the information are helped by the analyzed information to determine if they need to check on the person, make a call, connect to a video feed, etc.

Voice can be used as a unique identifier for each person. The software would separate the voice identities and the user can label them with the corresponding names and amount of talking. This input adds to the ability to track the presence of multiple individuals, their comings and goings (ex. guests, health workers, intruders). In addition, sounds like coughing and sneezing can be tracked for quantity and intensity (number of sneezes, mild, moderate or acute)

A library of sound clips can easily be built or purchased to detect everyday sounds such as eating using a metal silverware and a glass dish, breaking glass, doorbell, toilet flush, door opening/closing, faucet open, etc. The profile generator would alloy adding new sound clips to the library that may or may not be unique to the subject(s) and their environment.

Pet sounds such as dog barking can be used as an indication of interaction with people, strangers in the house, etc.

Video Software

In a similar way to voice, video analysis software can be used to detect situations as number of persons, identify them, detect falling objects, person in need of assistance, curtains closed/open, lights on, fire, etc.

Actions that can be Taken Based on Vitality Information.
1. Decide for appropriate moment to check on subject
   a. Initiate telephone or video call
   b. Dispatch someone else to help
2. Use patterns information to plan days
   a. Identify times of highs and lows of subject
3. Link causes to highs and lows of subject
   a. Remove negative triggers and increase positive influences
4. Send signals of awareness/remote presences/care to subject
   a. Use technology to communicate—tweet, e-cards
   b. Trigger avatar to specific actions—voice, visual, robotic, holographic avatars, etc.

The invention claimed is:

1. A system for automating the generation of health, safety and/or behavior information (wellness, activity level and/or environmental risk information) of a person or an animal, or a plurality of them (subject(s)), the system comprising:
a. A computing system
b. Input(s) collecting subject vitality related data from sensor(s) and/or smart device(s)
c. Output(s) sending subject vitality information to other systems
d. Networking capabilities
e. Storage capacity
f. A system configuration user interface

2. The system of claim 1, wherein the means for computing system include:
a. A computing device with hardware and software which could be stationary or mobile
b. Smart devices that are carried or worn by a subject
c. Virtual computing machine
d. A server computer
e. A database system
f. A plurality of computing machines
g. Cloud resources for computing with interfaces to other systems

3. The system of claim 1, wherein the means for computing system include:
a. Means for processing and analyzing data
b. Means for running software algorithms that qualify and/or quantify mood, body vitals, levels of activity and/or environmental safety factors (value domains)
c. Means for integrating two or more value domains of subject information for analysis
d. Means for entering subject or system specific profiles consisting of rules, set-points and thresholds
e. Means for generating a baseline from entered subject profile and system inputs
f. Means for comparing input information with baseline
g. Means for comparing input information with standard libraries
h. Means for building a custom library of events from input data
i. Means for detecting events as they happen
j. Means for asserting subject state of wellness, activity and environmental safety risks
k. Means for making subject assessments/diagnostics over a period of time from system data and historical information
l. Means for producing summary assertions from analysis

4. The system of claim 1, wherein the computing system qualifies and/or quantifies levels of activity of subject(s)

5. The system of claim 1, wherein the computing system qualifies and/or quantifies the mood of subject(s)

6. The system of claim 1, wherein the computing system qualifies and/or quantifies body vitals of subject(s)

7. The system of claim 1, wherein the computing system qualifies and/or quantifies environmental safety factors

8. A method for generating health, safety diagnostics, and/or identifying patterns of behavior of subject(s).

9. The method of claim 8 comprising rule(s) applied to system data.

10. The method of claim 8 comprising set-point(s) applied to system data.

11. The method of claim 8 comprising threshold(s) applied to system data.

12. The method of claim 8 comprising representative portions of subject data used as baseline to detect events or states of significance to the information recipients.

13. A method for generating vitality profiles for a person, animal or group. Vitality profiles are representative portions of subject data used as baseline to detect events or states of significance to the interested parties.
